(19) United States
(12) Patent Application Publication
(10) Pub. No.: US 2015/0273055 A1
(43) Pub. Date: Oct. 1, 2015

(54) METHODS AND COMPOSITIONS FOR EXPANDING HEMATOPOIETIC STEM AND PROGENITOR CELLS

(71) Applicant: UNIVERSITY OF ROCHESTER, Rochester, NY (US)

(72) Inventors: Julianne N.P. Smith, Rochester, NY (US); Laura M. Calvi, Rochester, NY (US)

(73) Assignee: UNIVERSITY OF ROCHESTER, Rochester, NY (US)

(21) Appl. No.: 14/432,788

(22) PCT Filed: Oct. 7, 2013
(86) PCT No.: PCT/US2013/063715

§ 371 Date: Apr. 1, 2015

Related U.S. Application Data
(60) Provisional application No. 61/710,173, filed on Oct. 5, 2012.

(51) Int. Cl. A61K 39/395; A61K 38/29; A61K 35/28; C07K 16/22

(52) U.S. Cl. CPC A61K 39/3955; C07K 16/22; A61K 38/29; A61K 35/28

(57) ABSTRACT

Provided herein are methods for increasing the number of hematopoietic stem and progenitor cells (HSPCs) in vivo or in vitro. The methods can include the steps of selecting a subject in need of an increased number of HSPCs and administering to the subject an effective amount of an inhibitor of VEGF to increase the number of HSPCs in the subject. Optionally, the methods include the steps of contacting a population of HSPCs and supporting cells with an inhibitor of VEGF, and contacting the population of HSPCs and supporting cells with a parathyroid hormone (PTH) receptor agonist. Also provided is a method for increasing the number of hematopoietic stem and progenitor cells (HSPCs) in a subject including the steps of administering to the subject an inhibitor of VEGF, and administering to the subject a parathyroid hormone (PTH) receptor agonist. The method is optionally used in a subject or for a with decreased osteogenesis, including, for example, when the subject has a bone fracture or has osteoporosis. Compositions and kits for increasing the number of HSPCs comprising (i) one or more doses of an inhibitor of VEGF; and (ii) one or more doses of a PTH receptor agonist are also provided.

### CROSS REFERENCE TO RELATED APPLICATIONS

This application claims the benefit of U.S. Provisional Application No. 61/710,173, filed on Oct. 5, 2012, which is incorporated by reference herein in its entirety.

### STATEMENT REGARDING FEDERALLY FUNDED RESEARCH

This invention was made with government support under Grant No. R01 DK081843 awarded by the National Institute of Health (NIH). The government has certain rights in the invention.

### BACKGROUND

Hematopoietic stem and progenitor cells (HSPCs) are primitive cells capable of regenerating all blood products throughout the life of an individual, balancing their self-renewal with progeny differentiation. These fundamental characteristics are in part intrinsic and in part conferred by the microenvironment or niche in which HSPCs reside. Studies in genetically altered animals have established osteoblastic cells as important members of the HSPC niche.

Hematopoietic stem and progenitor cells have therapeutic potential as a result of their capacity to restore blood and immune cells in transplant recipients. Furthermore, HSPCs have the potential to generate cells for other tissues such as brain, muscle and liver. Human autologous and allogeneic bone marrow transplantation methods are currently used as therapies for diseases such as leukemia, lymphoma, and other life-threatening diseases. For these procedures, a large amount of donor bone marrow must be isolated to ensure that there are enough HSPCs for engraftment.

### SUMMARY

Provided herein are methods for increasing the number of hematopoietic stem and progenitor cells (HSPCs). The methods include the steps of selecting a subject in need of an increased number of HSPCs, and administering to the subject an effective amount of an inhibitor of VEGF to increase the number of HSPCs in the subject. Optionally, the methods include the steps of contacting a population of HSPCs and supporting cells with an inhibitor of VEGF, and contacting the population of HSPCs and supporting cells with a parathyroid hormone (PTH) receptor agonist. Also provided is a method for increasing the number of HSPCs in a subject including the steps of administering to the subject an inhibitor of VEGF and administering to the subject a parathyroid hormone (PTH) receptor agonist. Compositions and kits for increasing the number of HSPCs comprising (i) one or more doses of an inhibitor of VEGF and (ii) one or more doses of a PTH receptor agonist are also provided. Optionally, the inhibitor of VEGF is bevacizumab.

The details of the method, compositions and kits are set forth in the accompanying drawings and the description below. Other features, objects, and advantages will be apparent from the description and drawings, and from the claims.

### DESCRIPTION OF DRAWINGS

FIG. 1A and 1B are pictures showing that activation of osteoblasts through systemic PTH treatment (FIG. 1A) or expression of a constitutively active PTH1 receptor (PTH1R) (FIG. 1B) increases trabecular bone and expands HSPCs in the bone marrow.

FIG. 2 is a graph showing an increase in HSPCs in the bone marrow through systemic PTH treatment.

FIGS. 3A-3H are graphs (FIGS. 3A-3G) and pictures (FIG. 3H) showing PTH increases bone marrow microvascularity. FIG. 3A is a graph showing histomorphometric analysis of bone marrow microvascular structures per hindlimb section (femur and tibia). FIG. 3B is a graph showing histomorphometric analysis of bone marrow vascular area, measured within a region of interest in the tibial metaphysis. FIG. 3C is a graph showing frequency of CD31+ Sca1+ bone marrow endothelial cells following TID PTH treatment. FIG. 3D is a graph showing frequency of CD31+ Sca1+ bone marrow endothelial cells isolated from CollcaPTH1R-expressing mice (TG) or wildtype littermates (WT). FIGS. 3E, 3F and 3G are graphs showing analysis of vascular micro-CT scans depicting number (FIG. 3E), volume (FIG. 3G) and spacing (FIG. 3F) between vascular structures larger than 20 um in diameter following TID (three times daily) PTH treatment (n=4 mice per group). FIG. 3H shows pictures of representative vascular micro-CT scans of trabecular blood vessels larger than 20 um in diameter located in the metaphysis of the femur of vehicle treated (top panels) and TID PTH treated mice (bottom panels). *P<0.05, **P<0.01, ***P<0.0001.

FIGS. 4A and 4B are pictures showing PTH increases VEGF-A protein within endosteal cells. FIG. 4A shows pictures of representative 10× images of the femur metaphysis from paraffin sections of vehicle treated (left panel) and TID PTH treated (right) mice immunohistochemically stained for VEGF-A in which VEGF-A protein is stained as indicated by the black arrows. FIG. 4B shows pictures of representative 20× images of VEGF-A stained sections shown in FIG. 4A, depicting the distribution of VEGF-A+ cells along endosteal surfaces as indicated by black arrowheads.

FIGS. 5A-5E are graphs showing PTH stimulates osteoblastic VEGF-A. FIG. 5A is a graph showing relative expression of VEGF-A in osteoblastic UMR106 cells at indicated times following PTH treatment. Levels are expressed as fold change over the expression in untreated (time 0 hours) UMR106 cells. FIG. 5B is a graph showing quantification of total UMR106 cell VEGF-A protein secreted into culture media by ELISA. FIG. 5C is a graph showing osteoblastic differentiation gene expression of MC3T3 cells at indicated durations of culture in mineralizing media. FIG. 5D is a graph showing relative expression of VEGF-A in osteoblastic MC3T3 cells 6 hours after PTH treatment at indicated days of osteoblastic differentiation. All expression levels are expressed as fold change over the expression in day 0 untreated cells. FIG. 5E is a graph showing quantification of total MC3T3 cell VEGF-A protein secreted into culture media by ELISA. **P<0.01; ***P<0.0001.

FIG. 6 shows pictures indicating PTH activation of osteoblasts increases regional bone marrow hypoxia. When administered to a live animal, pimonidazole-HCl precipitates in cells containing<14 μM oxygen or pO₂<10 mm Hg and can be detected immunohistochemically. FIG. 6 shows representative images of pimonidazole-HCl staining; which demonstrated increased regional hypoxia with osteoblastic PTH activation (bottom panels) as compared to wild type littermate controls (top panels) FIGS. 7A-7C show anti-VEGF therapy blocks endothelial expansion by PTH but does not reduce CD 105+ mesenchymal cells. FIG. 7A is a schematic of VEGF-blocking experiment. Mice were treated with PTH(1-34) or vehicle for 10 days thrice per day in combination with bevacizumab once per day and were sacrificed at day 11. FIG. 7B is a graph showing frequency of CD31+ Sca1+ bone marrow endothelial cells isolated from C57B1/6 mice treated as indicated. FIG. 7C is a graph showing frequency of

CD105+/Endoglin+ CD45− non-hematopoietic bone marrow mesenchymal cells isolated from C57B1/6 mice treated as indicated. *P<0.05, **P<0.01

FIGS. 8A-8E are graphs showing anti-VEGF therapy with or without PTH expands HSPCs. FIG. 8A is a graph showing frequency of multipotent progenitor/short term repopulating hematopoietic stem cells (MPP/ST-HSC) as defined immunophenotypically by flow cytometry from mice treated as indicated (see FIG. 7A). 2 independent experiments, n=6-7 mice per group. FIG. 8B is a graph showing analysis of peripheral blood after competitive transplantation as the percentage of donor cells in the total peripheral blood cell population in recipient mice at 3 weeks. FIGS. 8C, 8D, and 8E are graphs showing analysis of peripheral blood after competitive transplantation as the percentage of donor cells in the B220+ population (FIG. 8C), CD3+ population (FIG. 8D) and CD11b+ population (FIG. 8E) in recipient mice at 3 weeks. N=4 donors per treatment group, 10 recipients per donor group. *P<0.05, **P<0.005, ***P<0.0001

FIG. 9 shows that osteoblastic activation of the PTH receptor or systemic PTH increases marrow microvascularity. FIG. 9A shows representative images of H&E stained paraffin sections from vehicle treated (top panels) and TID PTH treated (bottom panels) mice. FIG. 9B shows histomorphometric analysis of bone marrow microvascular structures per hindlimb section (femur and tibia) as represented by FIG. 9A. FIG. 9C shows histomorphometric analysis of bone marrow vascular area, measured within a region of interest in the tibial metaphysis, represented by 9A. FIG. 9D show representative flow cytometry plots depicting the isolation of CD31+, Sca1+ bone marrow endothelial cells. FIG. 9E shows micrographs indicating the frequency of CD31+ Sca1+ bone marrow endothelial cells as defined by FIG. 9D following TID PTH treatment. FIG. 9F shows micrographs showing the frequency of CD31+ Sca1+ bone marrow endothelial cells defined by FIG. 9D isolated from CollcaPTH1R-expressing mice (TG) or their wildtype littermates (WT). FIG. 9G and H show micrographs of representative vascular micro-CT scans of trabecular blood vessels larger than 20 um in diameter located in the metaphysis of the femur of vehicle treated and TID PTH treated mice. FIG. 9I shows analysis of vascular micro-CT scans depicting number, volume and spacing between vascular structures larger than 20 um in diameter following TID PTH treatment (n=4 mice per group). **P<0.01, ***P<0.0001.

FIG. 10 shows that osteoblastic activation by PTH stimulates proangiogenic signals. FIG. 10A shows representative images of immunohistochemical staining for VEGF-A near trabeculae of the femur following TID PTH treatment. FIG. 10B shows relative Vegfa expression and secreted protein levels following PTH stimulation of UMR106 cells. FIG. 10C shows the number of vascular branch points in wild type and activated PTH1R animals. FIG. 10E shows the number of CD30+Sca1+ cells isolated from PTH TID or VEH treated mice. *p<0.01, **P<0.001, ***P<0.0001.

FIG. 11 shows that neither reduction in bone anabolism nor compensatory increase in angiogenic signaling occurs with PTH+anti-VEGF-A therapy. FIG. 11A shows the analysis of femoral trabecular bone parameters as calculated by microCT scan of hindlimbs harvested from mice treated with PTH and/or anti-VEGF-A therapy (αVEGF) as indicated. FIG. 11B shows the relative expression of angiogenic signals Vegfa and Fgf2 from osteolineage BM cells isolated from mice following TID PTH and anti VEGF therapy as indicated. FIG. 11D shows the relative expression of Vegfa within sorted, phenotypically defined HSPCs (LSKs) isolated from TID PTH and anti-VEGF therapy versus VEH controls. *P<0.05, **P<0.01, ***P<0.0001.

FIG. 12 shows that anti-VEGF-A treatment augments PTH-mediated expansion of HSPCs. FIG. 12A shows the frequency of hematopoietic stem and progenitor cells (Sca1+, c-Kit+ live BM cells not expressing hematopoietic lineage markers) (LSKs) by flow cytometry from mice treated as indicated. FIG. 12B shows the frequency of phenotypically-defined multipotent hematopoietic progenitors. FIG. 12C shows the frequency of phenotypically-defined long-term HSCs. FIG. 12D-E show the analysis of peripheral blood after competitive (1:4 donor:competitor) whole BM transplantation shows the percentage of donor-derived cells among blood lineages of primary (FIG. 12D) and secondary (FIG. 12E) recipients at indicated time points. Primary (FIG. 12F-G) and secondary (FIG. 12H-I) recipients were sacrificed at 25 weeks post transplant to analyze engraftment of donor-derived cells and HSPCs (LSKs) in the marrow. *P<0.05, **P<0.01, ***P<0.0001.

### DETAILED DESCRIPTION

Hematopoietic stem and progenitor cells (HSPCs) are primitive cells capable of regenerating all blood cells. During development, hematopoiesis translocates from the fetal liver to the bone marrow, which then remains the site of hematopoiesis throughout adulthood. Once hematopoiesis has been established in the bone marrow, the hematopoietic stem cells are not distributed randomly throughout the bone cavity. Instead, the hematopoietic stem cells are found in close proximity to the endosteal surfaces. The more mature stem cells (as measured by their CFU-C activity) increase in number as the distance from the bone surface increases. Finally, as the central longitudinal axis of the bone is approached, terminal differentiation of mature cells occurs. Given the relationship between the hematopoietic stem cells and the endosteal surfaces of the bone, it is thought that the osteoblast plays a role in hematopoiesis. Osteoblastic cells, for example, support the growth of primitive hematopoietic cells through the release of G-CSF and other growth factors.

Expanding the number of bone marrow derived stem cells is useful in transplantation and other therapies for hematologic and oncologic disease. As described in the methods herein, HSPC numbers are increased in vitro, ex vivo or in vivo. A method of increasing stem cell numbers in vivo reduces the time and discomfort associated with bone marrow/peripheral stem cell harvesting and increases the pool of stem cell donors. Currently, approximately 25% of autologous donor transplants are prohibited for lack of sufficient stem cells. In addition, less than 25% of patients in need of allogeneic transplant can find a histocompatible donor. Umbilical cord blood banks currently exist and cover the broad racial make-up of the general population, but these banks are currently restricted to use in children due to inadequate stem cell numbers in the specimens for adult recipients. A method to increase stem cell numbers permits cord blood to be useful for adult patients, thereby expanding the use of allogeneic transplantation.

Accordingly, a method for increasing the number of hematopoietic stem cells is provided. Specifically, provided is a method for increasing the number of hematopoietic stem and progenitor cells (HSPCs) in a subject. The method includes selecting a subject in need of an increased number of HSPCs and administering to the subject an effective amount of an inhibitor of VEGF to increase the number of HSPCs in the subject. Optionally, the inhibitor of VEGF is bevacizumab. Also provided is a method for increasing the number of hematopoietic stem and progenitor cells (HSPCs) in a subject including administering to the subject an inhibitor of VEGF, and administering to the subject a parathyroid hormone (PTH) receptor agonist. Optionally, the inhibitor of VEGF is bevacizumab.

Also provided is a method for increasing the number of hematopoietic stem and progenitor cells (HSPCs) including the step of contacting a population of HSPCs and supporting cells with an inhibitor of VEGF. Optionally, the inhibitor of VEGF is bevacizumab. Optionally, the method further includes contacting the population of HSPCs and supporting cells with a parathyroid hormone (PTH) receptor agonist. The contacting steps can occur in vitro, in vivo or ex vivo. The methods produce an increased number of HSPCs referred to herein as an increased population of HSPCs. As used herein an increased population of HSPCs refers to a population of HSPCs comprising at least one more HSPC, 10% more, 20% more, 30% more or greater as compared to the number of HSPCs prior to or in the substantial absence of administration of the provided agent or agents, e.g., bevacizumab and/or PTH. Also provided is a method of providing a population of HSPCs to a subject comprising administering to the subject the population of HSPCs made by the methods provided herein. The population of HSPCs is from the same subject or from a different subject. Optionally, the subject is a human. Optionally, the subject has depleted bone marrow or is a bone marrow transplant recipient.

Hematopoietic stem and progenitor cells (HSPCs) as used herein refer to cells having the capacity to self-renew and to differentiate into more mature blood cells comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages). HSPCs are interchangeably described as hematopoietic stem cells or hematopoietic progenitor cells throughout the specification. It is known that such cells may or may not include CD34⁺ cells. CD34⁺ cells are immature cells that express the CD34 cell surface marker. CD34+ cells are believed to include a subpopulation of cells with the stem cell properties defined above. Hematopoietic stem cells include pluripotent stem cells, multipotent stem cells (e.g., a lymphoid stem cell), and/or stem cells committed to specific hematopoietic lineages. The stem cells committed to specific hematopoietic lineages may be of T cell lineage, B cell lineage, dendritic cell lineage, Langerhans cell lineage and/or lymphoid tissue-specific macrophage cell lineage. Any of these HSPCs can be used in any of the methods described herein.

HSPCs are optionally obtained from blood products. A blood product includes a product obtained from the body or an organ of the body containing cells of hematopoietic origin. Such sources include unfractionated bone marrow, umbilical cord, peripheral blood, liver, thymus, lymph and spleen. All of the aforementioned crude or unfractionated blood products can be enriched for cells having hematopoietic stem cell characteristics in a number of ways. For example, the more mature, differentiated cells are selected against, via cell surface molecules they express. Optionally, the blood product is fractionated by selecting for CD34⁺ cells. CD34⁺ cells include a subpopulation of cells capable of self-renewal and pluripotentiality. Such selection is accomplished using, for example, commercially available magnetic anti-CD34 beads (Dynal, Lake Success, N.Y.). Unfractionated blood products are optionally obtained directly from a donor or retrieved from cryopreservative storage.

As used herein, the term “HSPC supporting cells” or “supporting cells” refers to cells naturally found in the vicinity of one or more HSPCs such that factors released by HSPC supporting cells reach the HSPCs by diffusion, for example. HSPC supporting cells include, but are not limited to, lymphoreticular stromal cells. Lymphoreticular stromal cells as used herein include, but are not limited to, all cell types present in a lymphoid tissue which are not lymphocytes or lymphocyte precursors or progenitors. Thus, lymphoreticular stromal cells include osteoblasts, epithelial cells, endothelial cells, mesothelial cells, dendritic cells, splenocytes and macrophages. Lymphoreticular stromal cells also include cells that would not ordinarily function as lymphoreticular stromal cells, such as fibroblasts, which have been genetically altered to secrete or express on their cell surface the factors necessary for the maintenance, growth or differentiation of hematopoietic stem cells, including their progeny. Lymphoreticular stromal cells are optionally derived from the disaggregation of a piece of lymphoid tissue. Such cells are capable of supporting in vitro or in vivo the maintenance, growth or differentiation of hematopoietic stem cells, including their progeny. By lymphoid tissue, it is meant to include bone marrow, peripheral blood (including mobilized peripheral blood), umbilical cord blood, placental blood, fetal liver, embryonic cells (including embryonic stem cells), aortal-gonadal-mesonephros derived cells, and lymphoid soft tissue. Lymphoid soft tissue as used herein includes, but is not limited to, tissues such as thymus, spleen, liver, lymph node, skin, tonsil, adenoids and Peyer's patch, and combinations thereof.

Lymphoreticular stromal cells provide the supporting microenvironment in the intact lymphoid tissue for the maintenance, growth or differentiation of hematopoietic stem cells, including their progeny. The microenvironment includes soluble and cell surface factors expressed by the various cell types which comprise the lymphoreticular stroma. Generally, the support which the lymphoreticular stromal cells provide is characterized as both contact-dependent and non-contact-dependent.

Lymphoreticular stromal cells, for example, are autologous (self) or non-autologous (non-self, e.g., heterologous, allogeneic, syngeneic or xenogeneic) with respect to hematopoietic stem cells. Autologous, as used herein, refers to cells from the same subject. Allogeneic, as used herein, refers to cells of the same species that differ genetically. Syngeneic, as used herein, refers to cells of a different subject that are genetically identical to the cell in comparison. Xenogeneic, as used herein, refers to cells of a different species. Lymphoreticular stroma cells are obtained, for example, from the lymphoid tissue of a human or a non-human subject at any time after the organ/tissue has developed to a stage (i.e., the maturation stage) at which it can support the maintenance, growth or differentiation of hematopoietic stem cells. The lymphoid tissue from which lymphoreticular stromal cells are derived usually determines the lineage-commitment hematopoietic stem cells undertake, resulting in the lineage-specificity of the differentiated progeny.

The co-culture of hematopoietic stem cells (and progeny thereof) with lymphoreticular stromal cells, usually occurs under conditions known in the art (e.g., temperature, CO₂ and O₂ content, nutritive media, duration, etc.). The time sufficient to increase the number of cells is a time that can be easily determined by a person skilled in the art and varies depending upon the original number of cells seeded. The amount of hematopoietic stem cells and lymphoreticular stromal cells initially introduced (and subsequently seeded) varies according to the needs of the experiment. The ideal amounts are easily determined by a person skilled in the art in accordance with needs.

As used throughout, by a subject is meant an individual. Thus, subjects include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject is optionally a mammal such as a primate or a human.

The subject referred to herein is, for example, a bone marrow donor or an individual with or at risk for depleted or limited blood cell levels. Optionally, the subject is a bone marrow donor prior to bone marrow harvesting or a bone marrow donor after bone marrow harvesting. The subject is optionally a recipient of a bone marrow transplant. The methods described herein are particularly useful in subjects that have limited or depleted bone marrow reserve such as elderly subjects or subjects prior to exposure or previously exposed to an immune depleting treatment such as chemotherapy. The subject, optionally, has a decreased blood cell level or is at risk for developing a decreased blood cell level as compared to a control blood cell level. As used herein the term control blood cell level refers to an average level of blood cells in a subject prior to or in the substantial absence of an event that changes blood cell levels in the subject. The event can be an event can be, for example, marrow failure syndrome, both pathogenic (for example, myelodysplastic syndrome, aplastic anemia, radiation and toxic marrow injury) as well as iatrogenic (for example, radiation and chemotherapy for cancers involving marrow sites, acute myelogenous and lymphocytic anemia, to name a few). Thus, an event that changes blood cell levels in a subject includes, for example, anemia trauma, chemotherapy, toxic marrow injury, bone marrow transplant and radiation therapy. For example, the subject has anemia or blood loss due to, for example, trauma. The provided agents, e.g., PTH and/or bevacizumab are administered to the subject, for example, before, at the same time, or after chemotherapy, radiation therapy or a bone marrow transplant. The subject optionally has depleted bone marrow related to, for example, congenital, genetic or acquired syndrome characterized by bone marrow loss or depleted bone marrow. Thus, the subject is optionally a subject that has a disease or disorder associated with bone marrow loss. Optionally, the disease or disorder is cancer or a disease or disorder that affects the immune system, such as, for example, HIV. Thus, the subject is optionally in need of hematopoeisis. Optionally, the subject is a bone marrow donor or is a subject with or at risk for bone marrow loss.

Hematopoietic stem cell manipulation is useful as a supplemental treatment to chemotherapy or radiation therapy. For example, hematopoietic stem cells are localized into the peripheral blood and then isolated from a subject that will undergo chemotherapy, and after the therapy the cells are returned. Thus, the subject is a subject undergoing or expected to undergo an immune cell depleting treatment such as chemotherapy, radiation therapy or serving as a donor for a bone marrow transplant. Bone marrow is one of the most prolific tissues in the body and is therefore often the organ that is initially damaged by chemotherapy drugs and radiation. The result is that blood cell production is rapidly destroyed during chemotherapy or radiation treatment, and chemotherapy or radiation must be terminated to allow the hematopoietic system to replenish the blood cell supplies before a patient is re-treated with chemotherapy. Therefore, as described herein, HSCs or blood cells made by the methods described herein are optionally administered to such subjects in need of additional blood cells.

Provided herein are methods of treating a subject with a disease or disorder associated with bone marrow loss. Optionally, the disease or disorder is anemia, cancer, or a disease or disorder that affects the immune system, such as, for example, HIV.

As shown herein, administration of an inhibitor of VEGF increases bone trabecular number and connectivity. Therefore, an inhibitor of VEGF can be used to increase bone strength, enhance fracture healing or enhance fracture protection. Optionally, the inhibitor of VEGF can be administered with a PTH receptor antagonist, for example, and not to be limiting, Teriparatide, which is a recombinant from of parathyroid hormone. Thus, in the treatment methods provided herein, the disease or disorder can be associated with decreased osteogenesis. For example, the disease or disorder associated with decrease osteogenesis can be, for example, osteoporosis, osteopenia, osteomalacia, osteodystrophy, osteoarthritis, osteomyeloma, arthritis, bone fracture, Paget's disease, osteogenesis imperfecta, bone sclerosis, aplastic bone disorder, humoral hypercalcemic myeloma, bone thinning following metastasis or hypercalcemia. A subject with decreased osteogenesis can also be a subject with a bone fracture.

The methods include the steps of selecting a subject with a disease or disorder associated with bone marrow loss and in need of an increased number of HSPCs and administering to the subject an effective amount of an inhibitor of VEGF to increase the number of HSPCs in the subject. Optionally, the inhibitor of VEGF is bevacizumab. Alternatively or additionally, the methods include the steps of administering to the subject an inhibitor of VEGF and administering to the subject a PTH receptor agonist. As used herein the terms treatment, treat, or treating refers to a method of reducing the effects of a disease or condition or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refers to an action, for example, administration of a therapeutic agent, that occurs before or at about the same time a subject begins to show one or more symptoms of the disease or disorder, which inhibits or delays onset or exacerbation of one or more symptoms of the disease or disorder. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

As used herein, a parathyroid hormone (PTH) receptor agonist includes, for example, PTH, derivatives of PTH, analogues of PTH and parathyroid related-protein (PTHrP). The term PTHrP includes PTHrP(1-36), which has been used in treatment of osteoporosis. PTHrP can be administered as an intermittent treatment (e.g., 500 μg, 625 μg, 750 μg daily) for 1-4 weeks. See, e.g., Horwitz et. Al., JCEM 95:1279 (2010), which is incorporated by reference herein in its entirety.

The term parathyroid hormone (PTH) encompasses naturally occurring human PTH, as well as synthetic or recombinant PTH (rPTH). Optionally, the PTH comprises SEQ ID NO:1. Production of recombinant human parathyroid hormone is described in EP Patent No. 0383751, which is incorporated by reference herein in its entirety. Further, the term parathyroid hormone encompasses full-length PTH as well as PTH fragments. Optionally, the PTH fragment comprises SEQ ID NO:2. It will thus be understood that fragments of PTH, in amounts giving equivalent biological activity to PTH, can be incorporated in the compositions described herein, if desired. Fragments of PTH incorporate at least the amino acid residues of PTH necessary for a biological activity similar to that of intact PTH. Examples of such fragments are PTH(1-31), PTH(1-34), PTH(1-36), PTH(1-37), PTH(1-38), PTH(1-41), PTH(28-48) and PTH(25-39).

Variants, derivatives and functional analogues of PTH are also useful in the provided methods, compositions and kits. Thus, the present application includes pharmaceutical formulations comprising such PTH variants, derivatives and functional analogues, carrying modifications like substitutions, deletions, insertions, inversions or cyclisations, but nevertheless having substantially the biological activities of parathyroid hormone. Stability-enhanced variants of PTH are known in the art from, e.g., WO 92/11286 and WO 93/20203, which are incorporated by reference herein in their entirety. Analogs of PTH include, but are not limited to, teriparatide. Variants of PTH can e.g. incorporate amino acid substitutions that improve PTH stability and half-life, such as the replacement of methionine residues at positions 8 and/or 18, and replacement of asparagine at position 16. Cyclized PTH analogues are disclosed in, e.g., WO 98/05683, which is incorporated by reference herein in its entirety. PTH receptor agonists are also described in, for example, International Publication No. WO2004/011484, which is incorporated by reference herein in its entirety.

Vascular endothelial growth factor (VEGF) is a protein produced by cells. VEGF stimulates vasculogenesis and angiogenesis. VEGF's normal function is to create new blood vessels during embryonic development or after injury or exercise. Overexpression of VEGF can contribute to diseases such as cancer. Thus, inhibitors of VEGF have been developed to control or slow progression of such diseases, e.g., cancer. Optionally, the VEGF is VEGF-A. Inhibitors of VEGF include, but are not limited to, antibodies. Bevacizumab is a humanized monoclonal antibody and was the first commercially available angiogenesis inhibitor. Its main action is the inhibition of the function of a natural protein called vascular endothelial growth factor (VEGF) that stimulates new blood vessel formation. Because most malignant tumors are highly dependent on angiogenesis it was expected that Bevacizumab can stop or delay growth of tumors. Bevacizumab binds directly to VEGF to form a protein complex which is incapable of further binding to VEGF receptor sites (which would initiate vessel growth) effectively reducing available VEGF. The Bevacizumab/VEGF complex is both metabolized and excreted directly.

As used herein, inhibitors of VEGF include inhibitors of VEGF-A, homologs, variants and iso-forms thereof. The amino acid and nucleic acid sequences of VEGF-A can be found at GenBank Accession Nos. NP_{—}001020537 and NM_{—}001025366. Thus, provided herein are inhibitors of VEGF-A for use in the provided methods. Inhibitors of VEGF-A include, but are not limited to, inhibitory peptides, drugs, functional nucleic acids and antibodies.

In addition, VEGF receptor antagonists would provide an additional strategy to expand HSPCs. Examples of VEGF receptor antagonists include, but are not limited to, lenvatinib, motesanib and pazopanib.

The provided agents, e.g., PTH receptor agonists and inhibitors of VEGF-A, are, optionally, formulated into compositions for administration in vitro or in vivo. Optionally, the compositions comprise one or more of the provided agents and a pharmaceutically acceptable carrier. Suitable carriers and their formulations are described in Remington: The Science and Practice of Pharmacy, 21^{st} Edition, David B. Troy, ed., Lippicott Williams & Wilkins (2005). Optionally, the compositions comprise one or more of the provided agents in a saline solution. By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject.

The compositions are administered in a number of ways depending on whether local or systemic treatment is desired, and on the area to be treated. The compositions are administered via any of several routes of administration, including topically, orally, parenterally, intravenously, intra-articularly, intraperitoneally, intramuscularly, subcutaneously, intracavity, transdermally, intrahepatically, intracranially, or nebulization/inhalation. Optionally, PTH is formulated for subcutaneous administration. Optionally, bevacizumab is formulated for intravenous administration.

Preparations for parenteral administration include sterile aqueous or non-aqueous solutions, suspensions, and emulsions. Examples of non-aqueous solvents are propylene glycol, polyethylene glycol, oils, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally used. Compositions can be formulated to provide quick, sustained or delayed release after administration by employing procedures known in the art. Certain carriers may be more preferable depending upon, for instance, the route of administration and concentration of composition being administered. Suitable formulations for use in the provided compositions can be found in Remington: The Science and Practice of Pharmacy, 21^{st} Edition, David B. Troy, ed., Lippicott Williams & Wilkins (2005).

Combinations of agents or compositions can be administered either concomitantly (e.g., as a mixture), separately but simultaneously (e.g., via separate intravenous lines) or sequentially (e.g., one agent is administered first followed by administration of the second agent). The term combination is used to refer to concomitant, simultaneous or sequential administration of two or more agents or compositions. Thus, bevacizumab can be administered concomitantly, simultaneously or sequentially with PTH.

According to the methods taught herein, the subject is administered an effective amount of one or more of the agents provided herein. The terms effective amount and effective dosage are used interchangeably. The term effective amount is defined as any amount necessary to produce a desired physiologic response (e.g., reduction of inflammation). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days, or weekly or bi-weekly for one or several weeks. Guidance can be found in the literature for appropriate dosages for given classes of pharmaceutical products. Optionally, the PTH is administered at a dose of 10, 20, 30, 40, 50, 60, 70, or 80 μg one or more times daily for one or several days. Optionally, 40 μg of PTH is administered one, two or three times daily as an infusion or other means of systemic administration. Optionally, the bevacizumab is administered at a dose of 0.1, 0.25, 0.5, 0.7, 1, 5, 10, 15 or more mg/kg. Optionally, bevacizumab is administered once daily, weekly, every two weeks or every three weeks. Optionally, the bevacizumab is administered as an infusion or other means of systemic administration.

Also provided herein is a pack or kit comprising one or more containers filled with one or more of the ingredients (e.g., ) described herein. Thus, for example, a kit described herein comprises (i) one or more doses of an inhibitor of VEGF, e.g., bevacizumab; and (ii) one or more doses of a PTH receptor agonist. Such kits optionally comprise solutions and buffers as needed or desired. The kit optionally includes a population of HSPCs made by the methods described herein, containers or compositions for making a population of HSPCs, administration means (e.g., a syringe, an IV bag, an infusion line, a pump, or the like) for administering the agents or the cells. Optionally associated with such pack(s) or kit(s) are instructions for use.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a composition is disclosed and discussed and a number of modifications that can be made to a number of molecules including the composition are discussed, each and every combination and permutation of the composition, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus , if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the materials for which they are cited are hereby specifically incorporated by reference in their entireties.

A number of embodiments have been described. Nevertheless, it will be understood that various modifications may be made. Accordingly, other embodiments are within the scope of the claims below.

### EXAMPLES

### Example 1

### Anti-VEGF Therapy With or Without Parathyroid Hormone Expands HSPCs

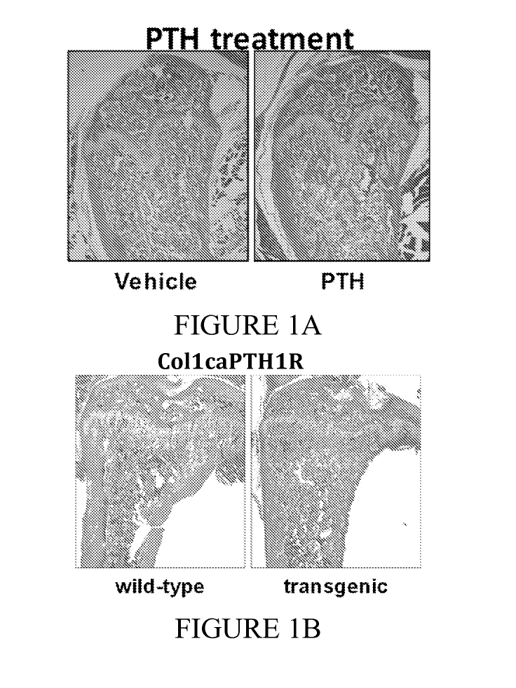

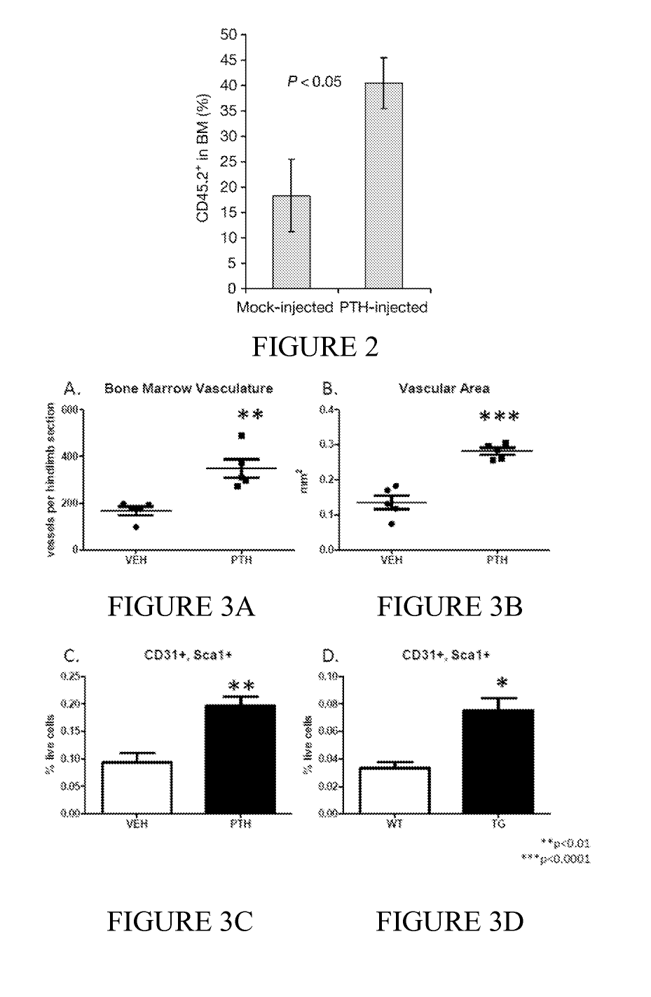

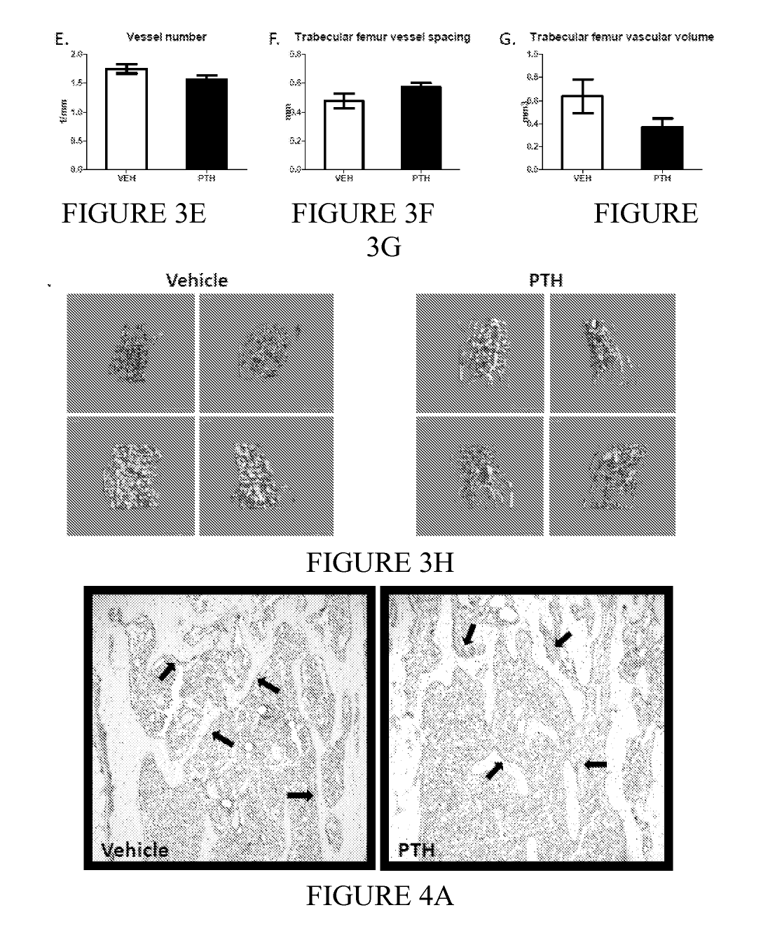

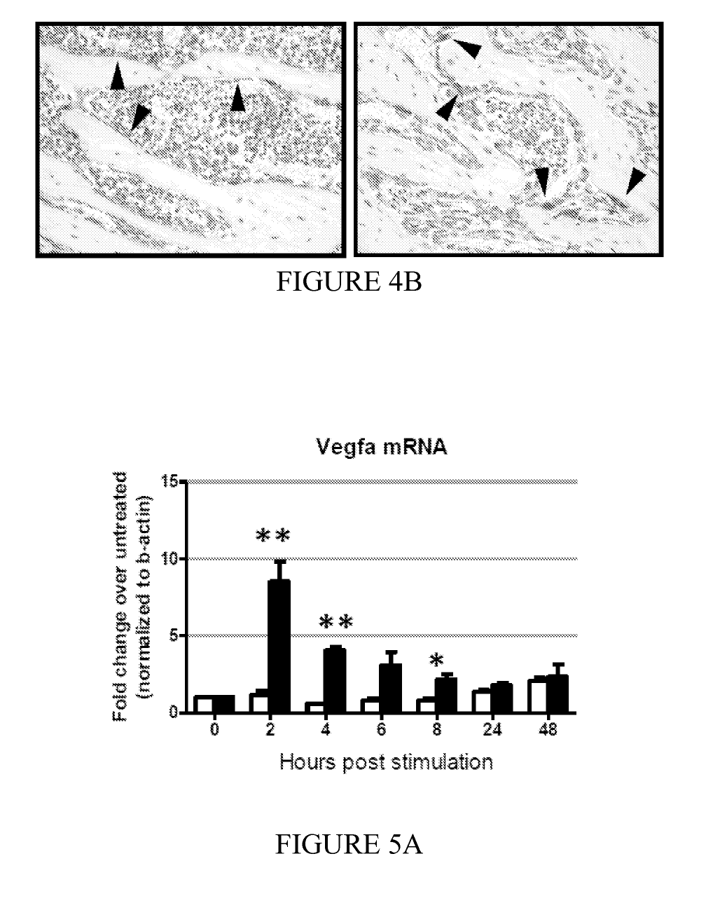

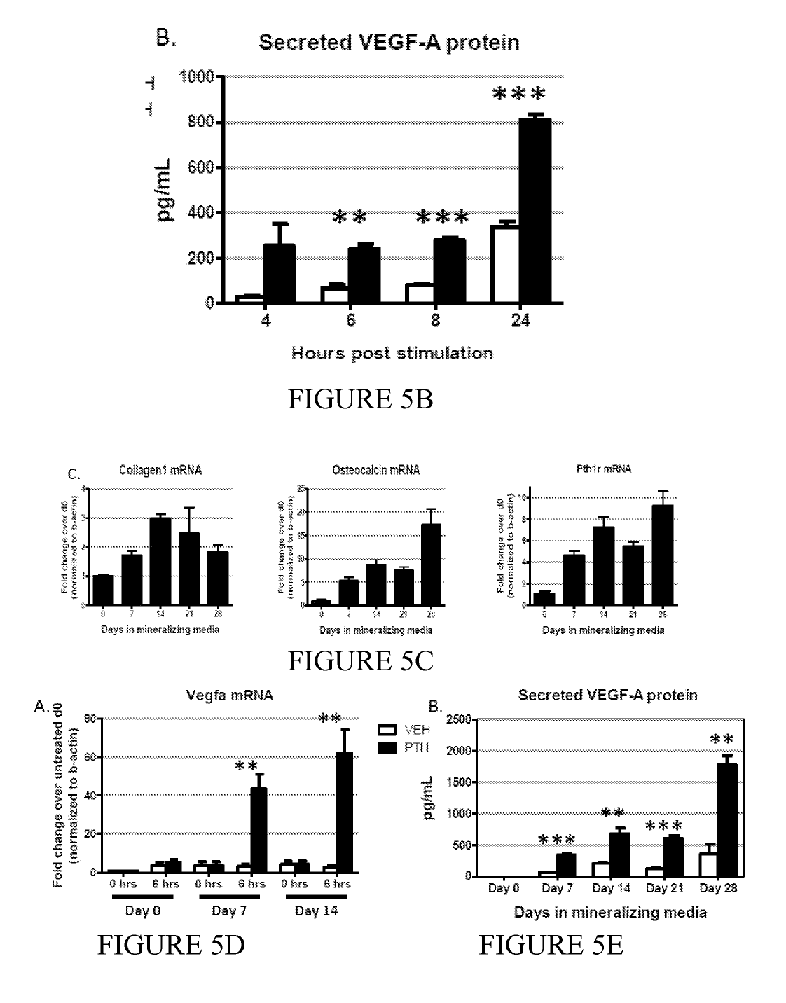

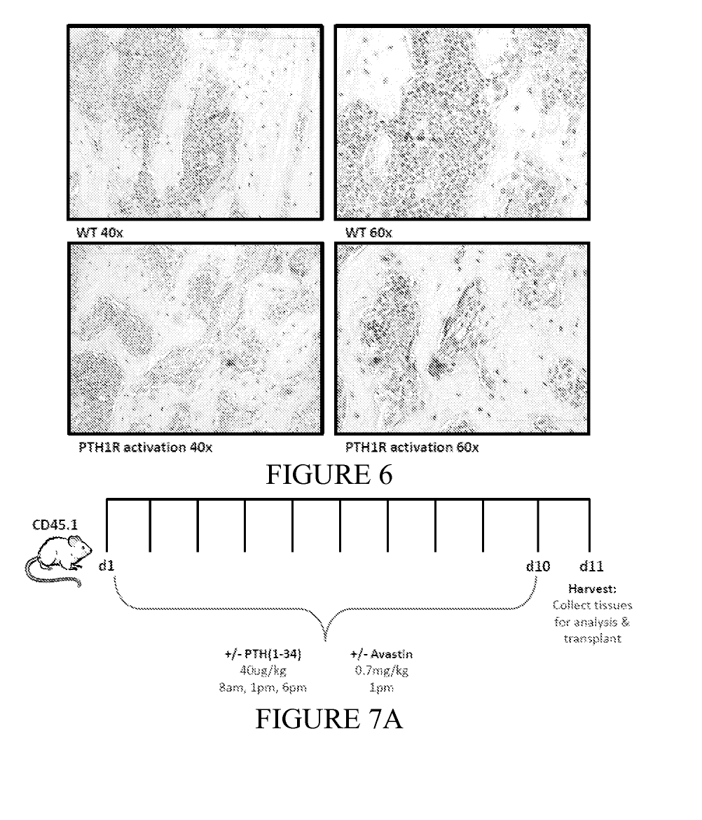

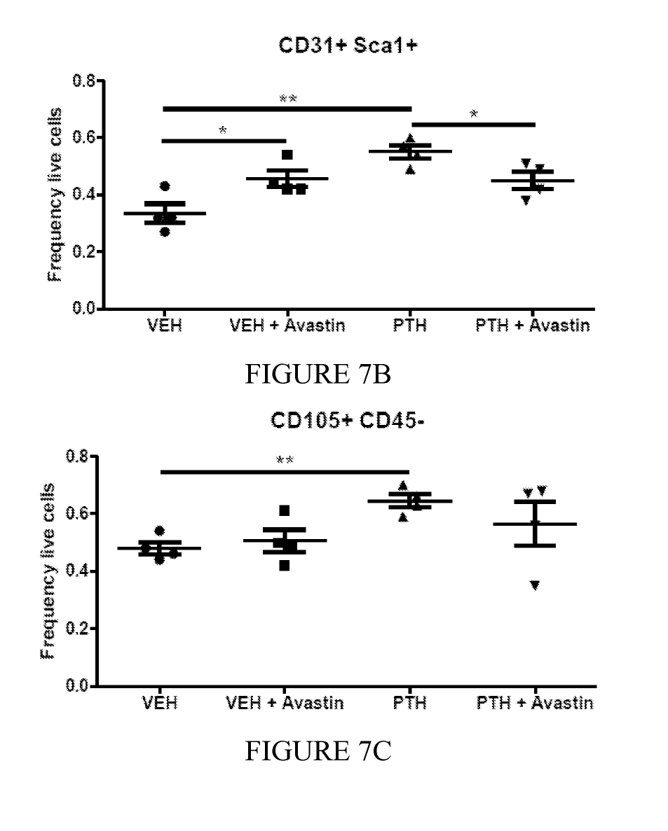

Osteoblasts (OBs) and the bone marrow (BM) vasculature constitute hematopoietic stem and progenitor cell (HSPC) niches. OB-activation by Parathyroid Hormone (PTH) expands HSPCs through the BM microenvironment. Activation of osteoblasts through systemic PTH treatment or expression of a constitutively active PTH1 receptor (PTH1R) increases trabecular bone and expands HSPCs in the bone marrow. See FIGS. 1A, 1B and FIG. 2. To test if the vasculature is modulated by PTH, it was first assessed whether PTH expands BM endothelial structures. In a murine model of HSPC-expanding PTH treatment, PTH increased microvessels and vascular area in hindlimb histology (FIG. 3A, 3B and 3E-3H). PTH also increased CD31+ Sca1+ BM endothelial cells (ECs) measured flow cytometrically (FIG. 3C and 3D). To determine if PTH expands BM vasculature by increasing proangiogenic signals, VEGF-A was analyzed in PTH-stimulated BM. Immunohistochemically, VEGF-A was increased along bone surfaces of PTH-treated mice (FIGS. 4A and 4B). To establish whether PTH directly stimulates osteoblastic Vegfa expression, mouse calvarial MC3T3 cells were treated with PTH at various stages of osteoblastic maturation. While differentiation did not alter baseline Vegfa, PTH strongly induced Vegfa in day 7 cells at two hours and was sustained 24 hours after treatment. The magnitude of induction increased throughout osteoblastic differentiation. PTH also increased secreted VEGF-A protein at corresponding time points. See FIGS. 5A-5E. Because the data implicate VEGF-A as a factor involved in vascular niche activation, the role of VEGF-A in PTH-induced vascular remodeling was investigated and the functional relevance of VEGF-A signaling in HSPC regulation was assessed using the anti-VEGF-A monoclonal antibody bevacizumab in combination with PTH. Bevacizumab blunted PTH-mediated expansion of phenotypic BM ECs when bevacizumab and PTH treatment was compared to PTH alone. See FIGS. 7B and 7C.

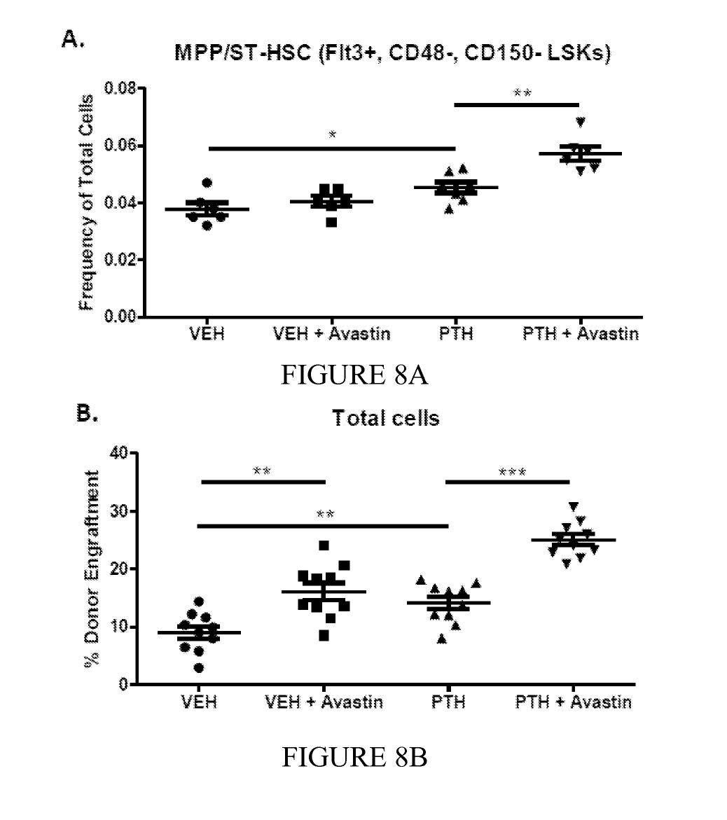

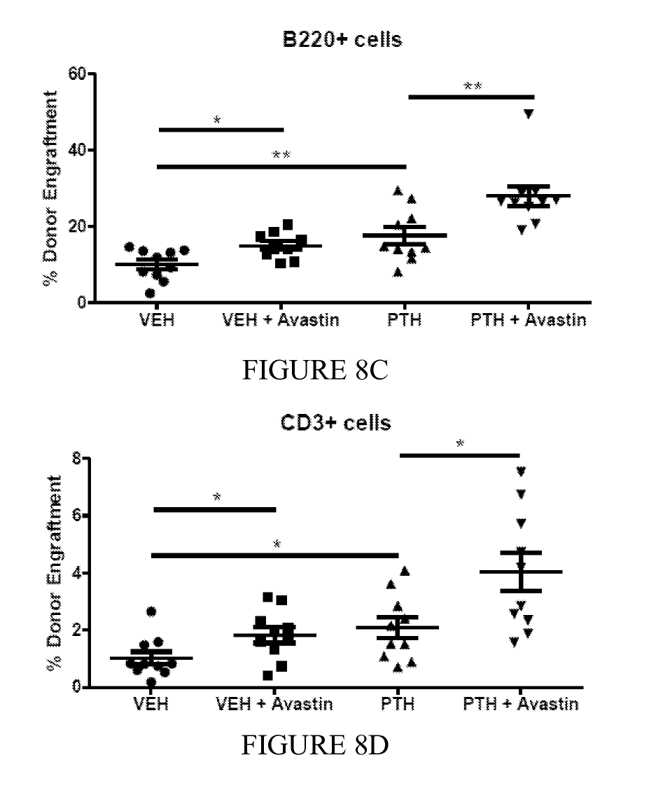

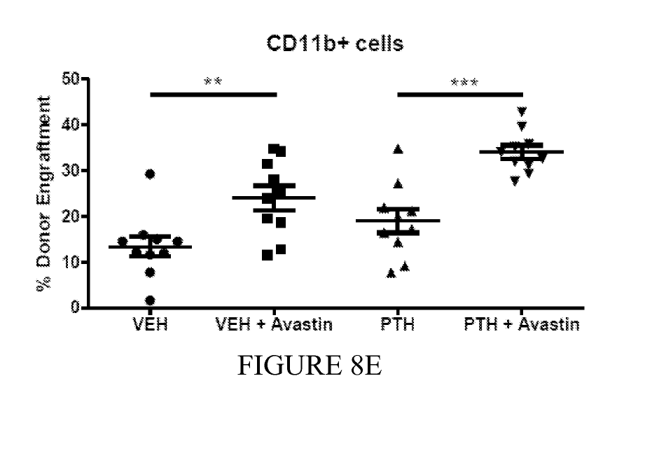

Furthermore, bevacizumab augmented PTH-induced expansion of phenotypically-identified multipotent progenitor/short term repopulating HSPCs (MPP/ST-HSPCs) when bevacizumab and PTH treatment was compared to PTH alone. 4 C57B1/6 CD45.1-expressing wildtype male mice (6-8 weeks of age) per group were treated with either (i) saline (Vehicle=VEH), (ii) saline plus 0.7 mg/kg bevacizumab diluted in saline (VEH + bevacizumab), (iii) 40 μg/kg Parathyroid hormone (1-34) diluted in saline (PTH) or (iv) 40 μg/kg PTH plus 0.7 mg/kg bevacizumab diluted in saline (PTH+bevacizumab). PTH or VEH were administered thrice per day for 10 consecutive days and 0.7 mg/kg bevacizumab was coadministered to the VEH+bevacizumab and PTH+bevacizumab groups once per day during the same 10 day interval. All drugs were administered by intraperitoneal injection. Following 10 days of treatment, mice were sacrificed (on the morning of the eleventh day) and samples were harvested. Hindlimb bone marrow samples were used to generate the data for FIG. 8A as well as for the competitive transplantation of whole bone marrow. Hindlimb bone marrow was harvested by crushing the right hindlimb of each mouse individually using a sterile mortar and pestle in 2.5 mL phosphate buffered saline, filtered through a 40 um cell strainer and placed on ice. For flow cytometric analysis, fresh, filtered bone marrow cells were subjected to a red blood cell lysis procedure using ammonium chloride and then prepared for flow cytometry by staining with a panel of fluorescently-conjugated antibodies recognizing markers of hematopoietic stem and progenitor cells. Samples were then run on an LSR-II 12 color flow cytometer and data was analyzed using TreeStar's FlowJo software. Statistics between treatment groups were analyzed by student's T test.

For FIGS. 8B, 8C, 8D, and 8E, the graphs of 3 week post transplantation analysis of peripheral blood, depicting experimental donor contribution to Total cells, B220+ B cells, CD3+ T cells and CD11b+ myeloid cells, a portion of the same bone marrow samples (not subjected to red blood cell lysis) was frozen in CryoStor media to allow for the future viable recovery of samples. At the time of transplant, frozen bone marrow samples were rapidly thawed and counted. Competitive transplantation of these cells was performed using a 4:1 donor to competitor cell ratio where the competitor was a C57B1/6 wildtype CD45.2-expressing male mouse and the recipient mice (10 mice per experimental group) were also C57B1/6 wildtype CD45.2-expressing male mice (6-8 weeks of age) that had been conditioned with a split dose of 10 Gray total body irradiation. Cell cocktails (containing the 4:1 ratio donor to competitor whole bone marrow cells) were transplanted into recipients by retro-orbital injection. Recipient mice were placed on a diet containing sulfatrim prior to the transplant and for 1 month post transplantation. Peripheral blood samples were collected at 3 weeks post transplant by bleeding from the sub-mandibular venous plexus into EDTA-containing microtainer blood tubes. Blood was then separated in order to isolate an enriched sample of mononuclear cells using PBS and dextran separation. Mononuclear cells were subjected to red blood cell lysis and then prepared for flow cytometry using a panel of antibodies recognizing B, T and myeloid cells as well as CD45.1 and CD45.2. Samples were then run on an LSR-II 12 color flow cytometer and data was analyzed using TreeStar's FlowJo software. Statistics between treatment groups were analyzed by student's T test. See FIGS. 8B-8E.

In summary, the data show PTH stimulation increases bone marrow microvascularity and endothelial populations. PTH directly stimulates osteoblastic expression of VEGF-A and may indirectly stimulate VEGF-A expression through the induction of bone marrow hypoxia. Inhibiting VEGF-A signaling blocks PTH-mediated expansion of bone marrow microvessels. Inhibiting VEGF-A signaling expands bone marrow HSPCs with at least short term hematopoietic repopulating ability. In conclusion, these data show that osteoblastic activation by PTH, although capable of increasing HSPCs, may limit the degree to which HSPC pools can expand through stimulation of pro-angiogenic signaling. Since both bevacizumab and the human analog of PTH are routinely used clinically and are well-tolerated, this combined approach offers a therapeutic strategy to expand HSPCs following bone marrow injury or in the treatment of hematopoietic malignancies.

### Example 2

Osteolineage cells (OBs) and the bone marrow (BM) vasculature constitute hematopoietic stem and progenitor cell (HSPC) niches. As set forth herein, OB-activation by Parathyroid Hormone (PTH) expands HSPCs. To test if the vasculature is also modulated by

PTH and whether PTH expands BM endothelial structures was examined. In a murine model of intermittent PTH treatment, PTH increased microvessels and vascular area in hindlimb BM as well as CD31+ Sca1+ BM endothelial cells (ECs). This PTH effect is OB-dependent since ECs were increased in transgenic mice with osteoblastic activation of the PTH receptor. PTH increased expression of the proangiogenic signal VEGF-A in OBs in vitro and strongly increased VEGF-A protein within endosteal cell populations in vivo. To determine the functional relevance of VEGF-A signaling in PTH-dependent microenvironmental activation and HSPC regulation, anti-VEGF-A monoclonal antibody bevacizumab (αVEGF) was used in combination with PTH in vivo. αVEGF significantly decreased the PTH-mediated expansion of ECs, restoring the number to control values, without blocking the bone anabolic action of PTH, indicating that PTH-dependent EC expansion in the BM is VEGF-A mediated. Surprisingly, αVEGF further increased the PTH-induced expansion of phenotypic and functional BM HSPCs. This effect persisted and was further enhanced by secondary transplantation. To examine whether co-administration of PTH and αVEGF expands long term HSPCs at the expense of hematopoietic progenitor and precursor maturation, acute hematopoietic recovery from chemotherapeutic injury was measured. The peripheral blood of PTH and αVEGF treated mice recovered similarly to that of controls and was restored to normal levels, indciating no impairment of the short term HSPC compartment. Together these data demonstrate that blocking VEGF-A in PTH-initiated vascular remodeling enhances expansion of long-term HSPCs. Both fibroblast growth factor 2 (FGF2) and VEGF-A are proangiogenic signals involved in bone remodeling. VEGF-A promotes endothelial sprouting and permeability while FGF2-stimulated blood vessels are less cell-permeable. Moreover, FGF2 expands marrow HSPCs. PTH simultaneously increased FGF2 and VEGF-A in OBs in vitro. αVEGF therapy permits dominance of PTH-induced FGF2 actions leading to preferential long-term HSPC expansion. These data also demonstrate the validity of niche manipulation for therapeutic targeting of specific HSPC subsets.

Since both PTH and bevacizumab are used clinically and are well-tolerated, this combined approach offers therapeutic strategies to expand HSPCs following bone marrow injury or in the treatment of hematopoietic malignancies.

Micro-CT Analysis

The limbs were scanned on a Viva CT 40 (Scanco Medical, Bruttisellen, Switzerland) using a 55-kVp, 145-uA current and a 300-ms integration time with a resolution of 12.5 μm. Trabecular analysis was conducted on a 1.25-mm region 50 μm above the growth plate in the femur and a 625-μm 50 um below the growth plate in the tibia. Cortical analysis was conducted 4.375 mm above/below the growth plate of the femur/tibia for a distance of 375 μm.

Osteoblastic Stimulation by PTH Remodels the BM Vasculature

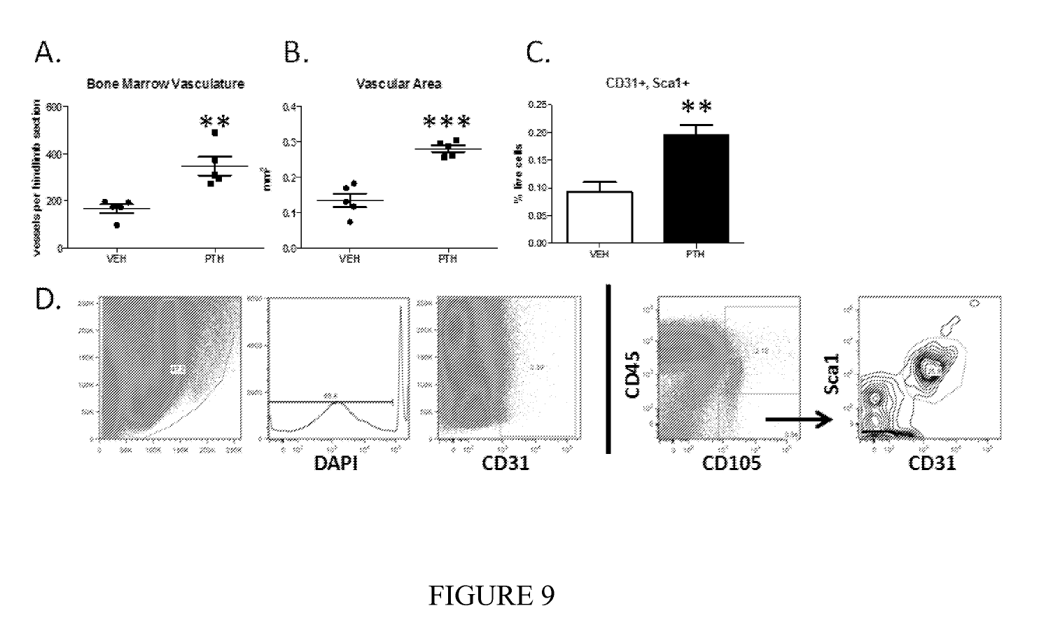

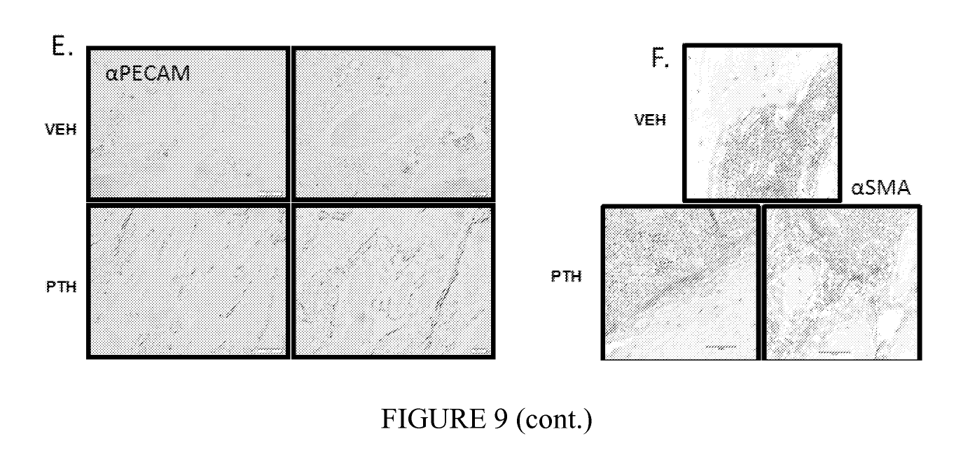

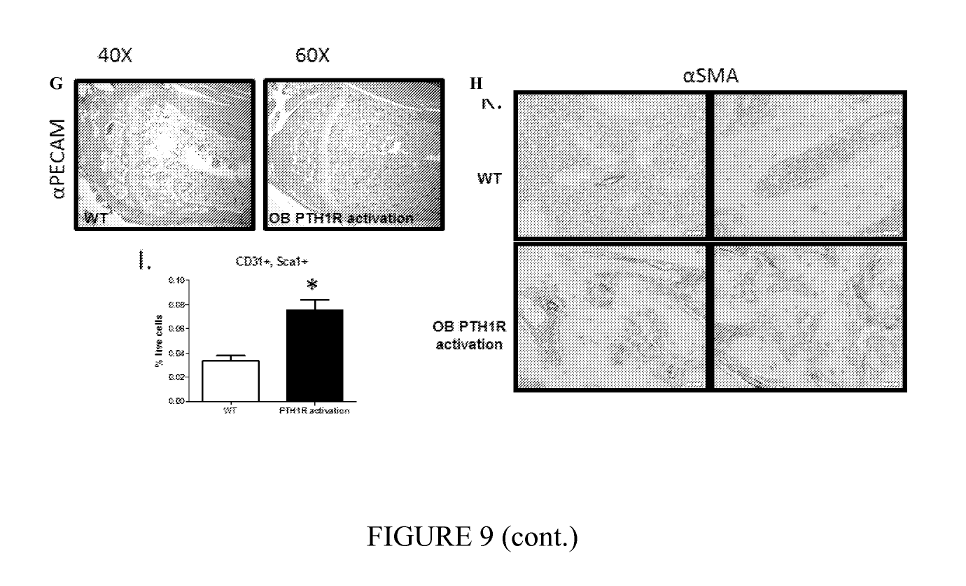

Methods of administration and analysis of the effects of PTH either alone or in combination are described above. To determine whether bone anabolic, HSPC-expanding regimens of PTH stimulation alter the vascular niche, mice were first treated intensively with PTH and the hindlimb BM vasculature was analyzed. PTH increased BM vascular structures per hindlimb section (femur and tibia) by histomorphometric analysis (FIG. 9A) as well as the area occupied by vasculature measured within the tibial metaphysis (FIG. 9B). To clarify whether intensive PTH treatment was vasodilatory to existing vessels or increasing de novo endothelial structures, BMCs of PTH-treated mice were analyzed phenotypically by flow cytometry (FIG. 9D). Intensive PTH treatment expanded populations of BM endothelial cells defined by this immunophenotyic signature (FIG. 9E). Moreover, no increase in vessel frequency, density or volume was observed when vessels larger than 20 um in diameter were examined using vascular uCT, indicating that the pro-angiogenic effects of PTH in the BM are confined to microvessels (<20 um in diameter). This expansion of small BM blood vessels was also observed in close association with both trabecular and cortical bone when CD31 immunostaining was examined in the BM (FIG. 9E). The PTH-induced increase in endothelial structures was accompanied by an increase in perivascular α smooth muscle actin (αSMA+) (FIG. 9F), indicating a concomitant increase in perivascular, HSPC supportive stromal cells.

It was then confirmed that the BM microvascularity following HSPC-expanding PTH treatment was indeed initiated by osteoblastic cells. To do so, the BM vasculature of mice genetically altered to express osteoblast-specific, constitutively active PTH/PTHrP receptors (collcaPTH1R mice), and shown to have increased trabecular bone and HSPC support (Calvi LM, Nature 425 (6960): 841-846 (2003)), was assessed. PTH1R stimulation of osteoblastic cells was sufficient to increase BM endothelial cell populations identified immunophenotypically (FIG. 9I) as well as CD31+ endothelial structures closely associated with bone (FIG. 9G) and αSMA+ perivascular stromal cells (FIG. 9H). Together these data confirm that, in the setting of bone anabolism and improved HSPC support, PTH also activates osteoblastic cells to remodel the BM microvasculature.

Osteoblastic Activation by PTH Stimulates Proangiogenic Signals

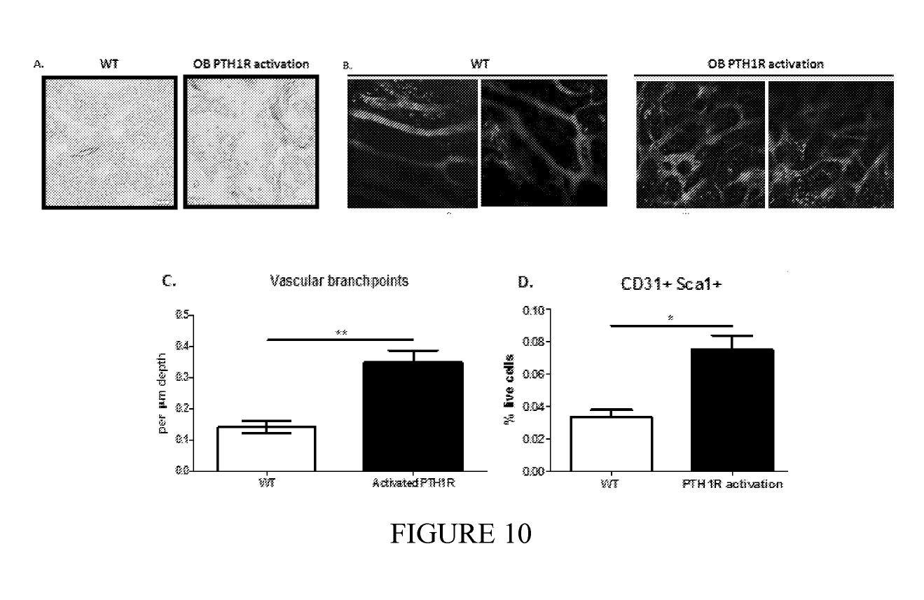

To determine whether the PTH-dependent increase in BM microvascularity is due to local induction of proangiogenic signals, immunostaining for candidate angiogenic factors already implicated in marrow endothelial signaling was performed. Immunohistochemical staining for VEGF-A in hindlimb BM of mice treated with TID PTH revealed a global increase in VEGF staining intensity, most dramatic along trabecular and cortical bone surfaces, as compared to vehicle treated controls (FIG. 10A). Representative photomicrographs at 40× demonstrate a heterogeneous distribution of VEGF staining intensity along endosteal surfaces following TID PTH as compared to vehicle treatment. These data indicate that populations of bone-lining stromal cells express VEGF in response to PTH and that a specific subset of cells may express and/or accumulate VEGF-A more robustly in response to PTH.

To determine if PTH increases osteoblast-derived VEGF-A, in vitro PTH stimulation experiments were performed on two osteoblastic cell lines. First, osteoblastic UMR106 cells were induced by PTH to express Vegfa at two hours post stimulation (1.133±0.2963 vs 8.500±1.320 fold change above baseline, n=3, p=0.0055) and demonstrated a corresponding increase in VEGF-A protein secretion by six hours post PTH-stimulation (FIG. 10B and 10C). Second, MC3T3-E1 cells, which possess osteoblastic differentiation potential, were also induced by PTH to express Vegfa in an effect that increased in magnitude throughout their osteoblastic differentiation and also corresponded to robust VEGF-A protein secretion. Osteoblastic differentiation was observed through maturational changes in gene expression of Collagen1, Osteocalcin and Pth1r and the ability to form mineralized bone nodules. These data indicate that osteoblastic cells, likely excluding immature osteoprogenitors or pre-osteoblasts, strongly produce VEGF-A in direct response to a single dose of PTH. To test whether PTH could directly induce VEGF-A in primary mouse osteoblastic cells as it could in osteoblastic cell lines, mouse bone marrow stroma was harvested and cultured in osteoblastic maturation media prior to stimulation with PTH at specific points throughout differentiation. Again, osteoblastic differentiation was monitored through analysis of Collagen1, Osteocalcin and Pth1r expression as well as by the formation of mineralizing bone nodules. When Vegfa gene expression was analyzed at various days of culture in mineralizing media and time points after PTH stimulation, increased Vegfa mRNA levels were observed as early as four hours post treatment of maturing osteoblastic cells (FIG. 10D), similar to what was observed in maturing MC3T3 cells treated with PTH. Surprisingly however, when soluble VEGF-A protein secreted into the culture media was examined, a significant effect of PTH treatment was not detected, in contrast to what was measured in PTH treated UMR106 or MC3T3 culture supernatant.

FGF2 is another angiogenic signal within the BM. In contrast to the effects of VEGF-A on endothelial permeability and migration, FGF2 enforces vascular stability. To determine whether osteoblastic activation by PTH can remodel the BM vasculature through the action of multiple angiogenic signals or VEGF-A alone, FGF2 changes in the marrow of PTH-stimulated mice were analyzed. Indeed, increased endosteal-associated FGF2 in hindlimb BM of mice treated with TID PTH, with a localization pattern similar to that of VEGF was observed. Moreover, FGF2 expression levels were increased within osteolineage cells isolated from TID PTH mice (FIG. 10E). These results demonstrate that HSPC-expanding PTH stimulation gives rise to multiple osteoblastic, bone-associated angiogenic signals.

Immunoreactive VEGF-A and FGF2 were both very high along endosteal surfaces of the hindlimb BM cavity following PTH as compared to control treatment. Direct, in vitro PTH stimulation of osteoblastic cells resulted in increased osteoblastic VEGF-A expression and protein secretion in both osteolineage immortalized cell lines as well as Vegfa expression in osteoblastic cells isolated from primary BM stroma. Moreover, osteolineage bone-associated cells isolated from mice treated with PTH demonstrated increases in Vegfa and Fgf2 expression. These results show that both in vitro and in vivo PTH stimulation increases proangiogenic factors VEGF and FGF2.

Anti-VEGF Treatment Neither Alters Osteoblastic Activation by PTH nor does it Directly Affect HSPC Survival

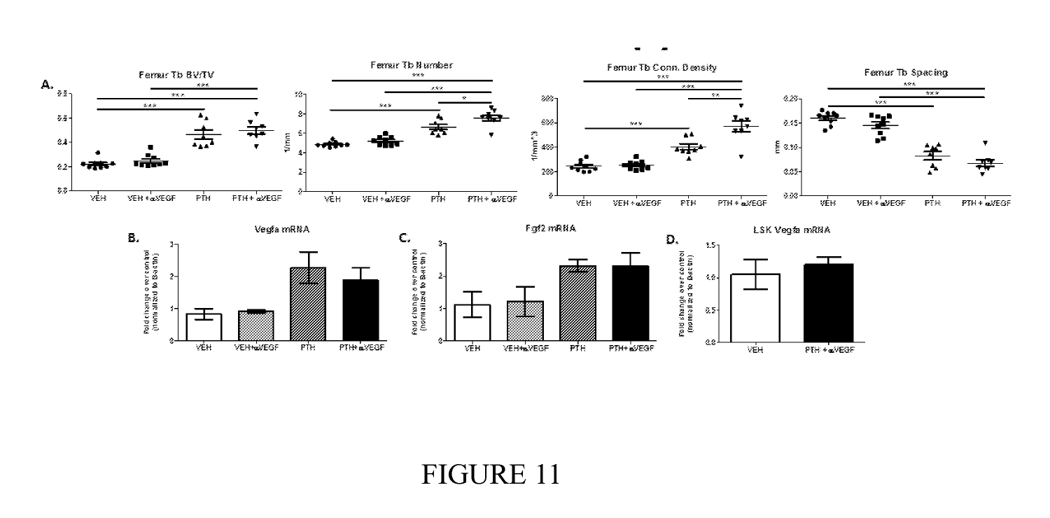

Due to the dramatic induction of osteoblastic VEGF-A observed in the setting of PTH stimulation, the functional relevance of this signal in multiple aspects of BM microenvironmental activation was analyzed. To assess the role of VEGF-A in a model of PTH-induced bone accrual, VEGF-A was inhibited using the anti-VEGF-A monoclonal antibody bevacizumab (hereon referred to as αVEGF) during TID PTH treatment in vivo. Administration of αVEGF did not significantly alter baseline or PTH-enhanced trabecular bone volume and trabecular spacing (FIG. 11A), however it did augment the effects of PTH on a number of bony trabeculae and connectivity density of the trabecular bone. These results indicate that therapeutic inhibition of VEGF-A does not abolish the effects of TID PTH on trabecular bone.

To rule out the possibility that αVEGF could act directly on HSPCs through an intracrine effect on VEGF-mediated cell survival, fresh hematopoietic progenitor-enriched lineage⁻, Sca-1⁺, c-kit⁺ BM cells (LSKs) were isolated from PTH+αVEGF treated mice and Vegfa expression levels were examined. Vegfa levels in LSKs from PTH+αVEGF treated mice were unchanged (FIG. 11D). Furthermore, the growth and survival of LSKs cultured in cytokine-supplemented media was not significantly affected by increasing concentrations of αVEGF. Together these data demonstrate that extracellular, osteoblast-derived VEGF-A is not required for the rapid trabecular bone remodeling stimulated by TID PTH or for the maintenance of intracellular VEGF survival signals in HSPCs.

VEGF Blockade During PTH Stimulation Remodels the BM Vascular Niche to be more Supportive of Long Term HSCs

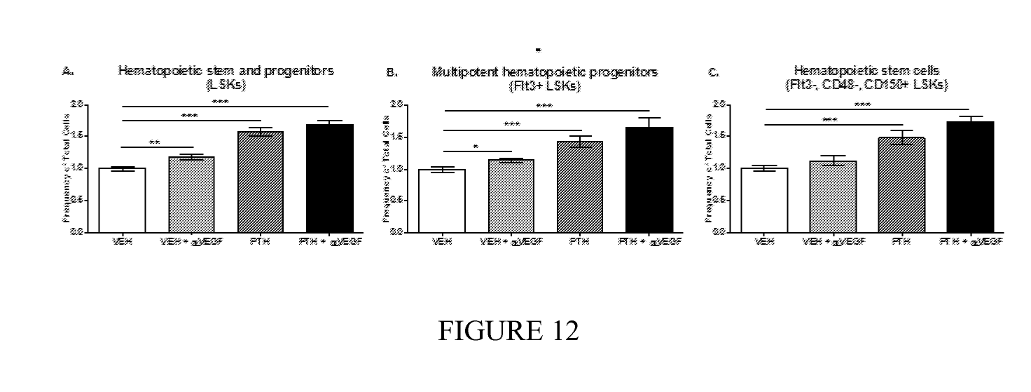

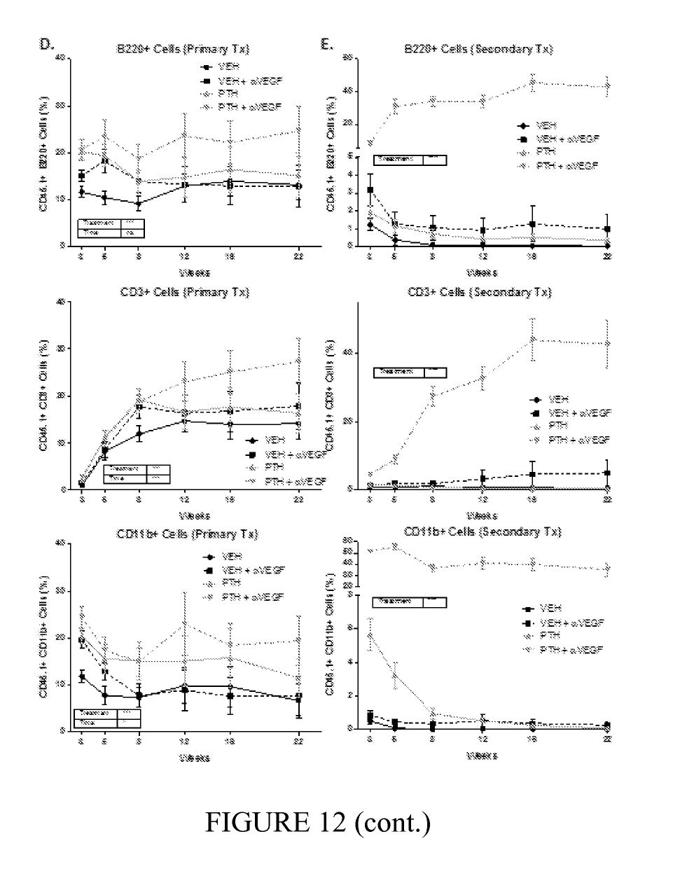

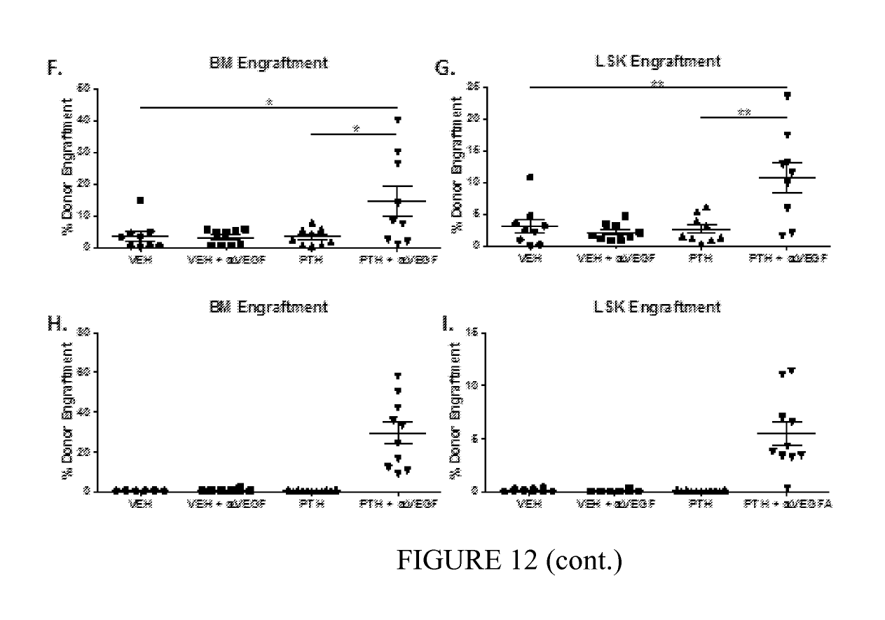

Based on the lack of an effect of αVEGF therapy on PTH mediated bone remodeling and on HSPC-intrinsic VEGF signaling, the role of microenvironmental VEGF signaling on HSPC regulation during niche activation by PTH was assessed. αVEGF treatment alone expanded hematopoietic progenitor populations prospectively identified as hematopoietic lineage−, Sca1+ and c-kit+ (LSKs) or by Flt3+, CD48−, CD150−, LSKs and similarly showed a trend towards an increase in the setting of PTH (FIG. 12A-B). VEGF inhibition also did not diminish PTH-mediated expansion of HSCs identified as Flt3−, CD48−, CD150+, LSKs (FIG. 12C). To assess the functional consequences of VEGF blockade with and without PTH stimulation, competitive transplantation of whole BM into lethally irradiated recipient mice was performed. While PTH and αVEGF therapies alone were capable of transiently increased multilineage hematopoietic reconstitution, recipients that received the combination PTH+αVEGF-treated BM demonstrated superior multilineage hematopoietic reconstitution throughout all time points evaluated (FIG. 4D-E). This effect was more dramatic and sustained than predicted phenotypically (FIG. 4A-C), indicating that PTH+αVEGF can alter HSPC behavior rather than absolute number within the BM.

To examine the effects of αVEGF therapy on long-term self-renewing HSPCs, secondary transplantation was performed 25 weeks after primary transplantation. At the time of transplantation, the level of donor-derived BM LSKs was greatly increased in mice that received PTH+αVEGF-treated BM over that of vehicle or PTH alone (FIG. 4F-G). Peripheral blood of secondary recipients transplanted with PTH+αVEGF BM showed robustly increased hemapoietic repopulation in all compartments and at all time points analyzed (FIG. 4E), demonstrating expansion of long-term HSC activity. To determine whether this dramatic increase in long-term stem cell function occurs at the expense of hematopoietic progenitor cell activity, PTH+αVEGF mice were exposed to acute hematopoietic injury by the chemotherapeutic agent 5-Fluorouracil and short term recovery was analyzed. PTH+αVEGF treated mice recovered fully from the injury and the kinetics of their recovery were the same as those of vehicle treated controls. Together these data indicate that by specifically inhibiting the VEGF component of microenvironmental activation by PTH, improved long-term HSPCs expansion can be achieved.

Sequence Listing

SEQ ID NO: 1 PTH (84 aa)
svseiqlmhnlgkhlnsmervewlrkklqdvhnfvalgaplaprdagsqr
prkkednvlveshekslgeadkadvnvltkaksq
SEQ ID NO: 2 PTH (1-34 aa)
svseiqlmhnlgkhlnsmervewlrkklqdvhnf

1. A method for increasing the number of hematopoietic stem and progenitor cells (HSPCs) in a subject comprising
(a) selecting a subject in need of an increased number of HSPCs; and
(b) administering to the subject an effective amount of an inhibitor of VEGF-A to increase the number of HSPCs in the subject.

2. A method for increasing the number of hematopoietic stem and progenitor cells (HSPCs) in a subject comprising
(a) administering to the subject an inhibitor of VEGF-A; and
(b) administering to the subject a parathyroid hormone (PTH) receptor agonist.

3. The method of claim 2, wherein the PTH receptor agonist is parathyroid hormone (PTH) or a derivative or analog thereof.

4. (canceled)

5. (canceled)

6. (canceled)

7. (canceled)

8. (canceled)

9. (canceled)

10. The method of claim 1, wherein the subject is a bone marrow donor.

11. The method of claim 1, wherein the subject has depleted bone marrow.

12. The method of claim 1, wherein the subject is a bone marrow transplant recipient.

13. The method of claim 1, wherein the subject has anemia.

14. The method of claim 1, wherein the subject has a disease or disorder associated with bone marrow loss.

15. (canceled)

16. (canceled)

17. The method of claim 1, wherein the subject has been treated with a chemotherapeutic agent or radiation.

18. A method for increasing the number of hematopoietic stem and progenitor cells (HSPCs) comprising
(a) contacting a population of HSPCs and supporting cells with an inhibitor of VEGF-A; and
(b) contacting the population of HSPCs and supporting cells with a parathyroid hormone (PTH) receptor agonist.

19. The method of claim 18, wherein the PTH receptor agonist is parathyroid hormone (PTH) or a derivative or analog thereof.

20. (canceled)

21. (canceled)

22. (canceled)

23. (canceled)

24. (canceled)

25. The method of claim 18, wherein the PTH receptor is located on one or more of the supporting cells.

26. The method of claim 18, wherein the supporting cells comprise lymphoreticular stromal cells or osteoblasts.

27. (canceled)

28. (canceled)

29. (canceled)

30. A method of providing a population of HSPCs to a subject comprising administering to the subject the population of HSPCs made by the method of claim 18.

31. The method of claim 30, wherein the population of HSPCs are from the same subject.

32. The method of claim 30, wherein the population of HSPCs are from a different subject.

33. (canceled)

34. The method of claim 30, wherein the subject has depleted bone marrow.

35. The method of claim 30, wherein the subject is a bone marrow transplant recipient.

36. (canceled)

37. (canceled)

38. (canceled)


SEQUENCE LISTING (2 sequences)

SEQ ID NO: 1 - protein, 84 aa, Homo sapiens
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 2 - protein, 34 aa, Homo sapiens
SVSEIQLMHNLGKHLNSMERVEWLRKKLQDVHNF